(19) United States
(12) Patent Application Publication
(10) Pub. No.: US 2023/0068456 A1
(43) Pub. Date: Mar. 2, 2023

(54) REAL-TIME MONITORING OF IN VIVO FREE RADICAL SCAVENGERS THROUGH HYPERPOLARIZED N-ACETYL CYSTEINE ISOTOPES

(71) Applicant: THE UNITED STATES OF AMERICA, AS REPRESENTED BY THE SECRETARY, DEPARTMENT OF HEALTH AND HUMAN SERVIC, Bethesda, MD (US)

(72) Inventors: Ana Christina Opina, Gaithersburg, MD (US); Murali Krishna Cherukuri, Potomac, MD (US); Rolf Eric Swenson, Silver Spring, MD (US); Kazutoshi Yamamoto, North Bethesda, MD (US); Deepak Sail, Potomac, MD (US)

(21) Appl. No.: 17/793,083

(22) PCT Filed: Jan. 15, 2021
(86) PCT No.: PCT/US2021/013634

§ 371 Date: Jul. 15, 2022

Related U.S. Application Data
(60) Provisional application No. 62/961,855, filed on Jan. 16, 2020.

(51) Int. Cl. A61K 49/10; A61K 45/06; A61P 35/00

(52) U.S. Cl. CPC A61K 49/10; A61K 45/06; A61P 35/00

(57) ABSTRACT

A method of diagnosing or monitoring a patient suffering from cancer, the method comprising: administering a pharmaceutical composition comprising an effective amount of an active agent, wherein the active agent is [1-¹³C] N-acetyl cysteine, a deuterated derivative thereof, a pharmaceutically acceptable salt of any of the foregoing thereof, or a combination thereof, together with a pharmaceutically acceptable carrier to the patient; and diagnosing or monitoring the patient by hyperpolarized ¹³C-MRI. Also disclosed is a method of synthesizing [1-¹³C] N-acetyl cysteine or a deuterated derivative thereof.

### CROSS REFERENCE TO RELATED APPLICATIONS

This application claims priority from U.S. Provisional Application No. 62/961,855, filed 16 Jan. 2020, which is hereby incorporated by reference in its entirety.

### STATEMENT OF GOVERNMENT SUPPORT

This invention was made in part with government support from the National Institutes of Health. The government has certain rights in this invention.

### BACKGROUND OF THE INVENTION

### 1. Field of the Invention

The present invention is directed to [1-¹³C] N-acetyl cysteine and deuterated derivatives thereof as well as their use as an imaging, therapeutic, or diagnostic agent and methods for preparation.

### 2. Brief Description of the Related Art

N-acetyl cysteine (NAC), the acetylated derivative of the amino acid L-cysteine, is a precursor of glutathione. Glutathione elevates detoxification and works directly as a free radical scavenger. NAC also elevates glutathione-S-transferase activity, promotes liver detoxification, and also acts directly as a free radical scavenger. NAC is used as a therapeutic and involved to stimulate glutathione synthesis and potential therapeutic agent in the treatment of cancer, heart disease, HIV infection, and other diseases related to oxidative stress. NAC stimulates glutathione synthesis, since cysteine supply appears to be the rate-limiting step in the glutathione synthesis.

Cells normally exist in a fine balance between reductive and oxidative states. When this balance is disrupted, either by external environmental stimuli or by abnormal metabolic states, the cellular integrity is compromised. To maintain the oxidative balance, the cells employ a variety of compartmentalized antioxidant systems to eliminate reactive oxygen species before damage can occur. Chief among these is glutathione/glutathione disulfide (GSH/GSSG) redox pair, which serves to maintain thiol redox balance through the NADPH-dependent reduction of glutathione disulfide (GSSG), and also serves as a primary control point in the coupled reactions that maintain intracellular redox balance. In general, imbalance of redox state is also closely linked to the genesis and progression of numerous pathological conditions, including cancer, aging, diabetes, obesity, neurodegeneration, age-related retinopathy, cochlear degeneration, and chronic inflammatory diseases.

Particularly, malignant tumors frequently accumulate large amounts of glutathione as a countermeasure, because the high rate of aerobic glycolysis found in many cancers can result in oxidative stress.

Therefore, there is a strong interest in determining the GSH/GSSG balance in vivo. Furthermore, imaging redox environment of GSH/GSSH balance can be a powerful diagnostic strategy for non-invasively detecting cancer tissues, in particular, and assessing their early readout of therapeutic responses for ionizing radiation and some pharmaceuticals. Measurements are complicated by the fact that glutathione is primarily intracellular and likely varies within a tumor due to metabolic heterogeneity. ¹³C labeled dehydroascorbic acid has been used to probe the GSH/GSSG balance indirectly in preclinical studies, however, the pancreatic toxicity associated with dehydroascorbic acid may limit its in vivo and clinical use further. There is a need to develop suitable precursor of glutathione as a reliable probe to monitor redox status in order to diagnose or treat diseases such as, for example, cancers. The present invention is believed to be an answer to that need.

### SUMMARY OF THE INVENTION

In one aspect, the present invention is directed to a method of diagnosing or monitoring a patient suffering from cancer, the method comprising (1) administering a pharmaceutical composition comprising an effective amount of an active agent, wherein the active agent is [1-¹³C] N-acetyl cysteine, a deuterated derivative thereof, a pharmaceutically acceptable salt of any of the foregoing thereof, or a combination thereof, together with a pharmaceutically acceptable carrier to the patient; and (2) diagnosing or monitoring the patient by hyperpolarized ¹³C-MRI.

In another aspect, the present invention is directed to a method of synthesizing [1-¹³C] N-acetyl cysteine or a deuterated derivative thereof, the method comprising reacting a [1-¹³C]-cysteine or a deuterated derivative thereof with an acetylating agent to form a [1-¹³C]N-acetyl cysteine or a deuterated derivative thereof; and isolating said [1-¹³C] N-acetyl cysteine or a deuterated derivative thereof.

These and other aspects will become apparent from the following detailed description and drawings.

### BRIEF DESCRIPTION OF THE DRAWINGS

The following Detailed Description, given by way of Examples, but not intended to limit the invention to specific embodiments described, may be understood in conjunction with the accompanying figures, in which:

FIG. 1A shows synthetic scheme of [1-¹³C] NAC;

FIG. 1B shows a hyperpolarization build-up curves of [1-¹³C] NAC showing the drastic improvement of polarization using the optimized condition of a NaOH solution vs conventional DMSO solutions;

FIG. 1C ¹³C NMR spectra of unlabeled NAC at 1T NMR confirm the pH dependence of polarization, an asterisk (*) is from a referencing standard of ¹³C Urea;

FIG. 1D is a dynamic spectra of hyperpolarized [1-¹³C] NAC at 3T MRI indicates a T₁ relaxation time of 19.6 seconds;

FIG. 2A is a in cell dynamic ¹³C NMR spectra of hyperpolarized [1-¹³C] NAC at 1T NMR on 20×10⁶ cells of human pancreatic tumor cell lines of Hs766t (left) and expanded spectra with 100 times magnifications at 2 seconds after the hyperpolarized [1-¹³C] NAC injections in Hs766t (right);

FIG. 2B is a in cell dynamic ¹³C NMR spectra of hyperpolarized [1-¹³C] NAC at 1T NMR on 20×10⁶ cells of human pancreatic tumor cell lines of SU.86.86 (left) and expanded spectra with 100 times magnifications at 2 seconds after the hyperpolarized [1-¹³C] NAC injections in SU.86.86 (right);

FIG. 2C shows a graph of NAC-GSH/NAC ratio versus Time illustrating time dependence of NAC-GSH/NAC peak intensity ratio after mixing HP-NAC with PDAC cells;

FIG. 2D shows a comparison of the ratios of NAC-GSH to NAC between Hs766t and SU.86.86 cell lines;

FIG. 3A shows an ESI-MS spectra of SU.86.86 tumor extracts with (top) and without (middle) isotope labeling in NAC, and a high energy ESI-MS spectrum of NAC-GSH with possible fragment identifications (bottom);

FIG. 3B shows a ¹³C NMR spectra of synthesized model compounds that represent potential products in comparison to the spectrum from the hyperpolarized [1-¹³C] NAC MRI experiments in Hs766t tumor xenograft at 20 seconds after the intravenous (i.v.) injection (top);

FIG. 4A shows a dynamic ¹³C MR spectra of hyperpolarized [1-¹³C] NAC at 3T MRI on human pancreatic tumor xenografts of Hs766t;

FIG. 4B shows a Dynamic ¹³C MR spectra of hyperpolarized [1-¹³C] NAC at 3T MRI on human pancreatic tumor xenografts of SU.86.86;

FIG. 4C shows a graph of NAC-GSH/NAC ratio versus Time after HP-NAC i.v. injections in seconds illustrating differences in the conversions reflect the redox status of each tumor;

FIG. 4D shows a comparison of the ratios of NAC-GSH to NAC between Hs766t and SU.86.86 tumor xenografts;

FIG. 4E shows a site-specific differences in chemical conversions of hyperpolarized [1-¹³C] NAC by ¹³C Chemical shift imaging in Hs766t xenografts;

FIGS. 5A and 5B shows a dynamic ¹³C MR spectra of hyperpolarized natural abundance NAC on 1T NMR spectrometer illustrating that an only [1-¹³C] NAC signal can be observed in the hyperpolarized ¹³C NMR spectra, although NAC has two carbonyl groups in the chemical structure, including [1-¹³C] and [4-¹³C] (FIG. 5A), as shown in the thermal NMR of natural abundance NAC at 1T NMR spectrometer (FIG. 5B);

FIGS. 6A and 6B shows a broad distribution of hyperpolarized [1-¹³C] NAC in the mouse body observed by ¹³C Chemical Shift Imaging (CSI) (FIG. 6A), a hyperpolarized ¹³C CSI in the mouse head acquired within 30 seconds after the injection of hyperpolarized [1-¹³C] NAC (FIG. 6B);

FIGS. 7A and 7B shows a hyperpolarized ¹³C NAC enzymatic assays with acylase 1 without (FIG. 7A) and with an inhibitor, n-Butylmalonic acid (FIG. 7B) on 1T NMR spectrometer;

FIG. 8 shows a dynamic ¹³C MRI of hyperpolarized [1-¹³C] NAC on 1T NMR spectrometer;

FIG. 9A shows a dynamic ¹³C MRI of hyperpolarized [1-¹³C] NAC-[1-¹³C] NAC at pH=7 on 1T NMR spectrometer (A);

FIG. 9B shows a thermal spectra of [1-¹³C] NAC-[1-¹³C] NAC was observed on 1T NMR with 16384 scans, 5 mM ProHance (B);

FIG. 9C shows a decay of dynamic ¹³C MR signal of hyperpolarized [1-¹³C] NAC-[1-¹³C] NAC on 1T NMR spectrometer (C);

FIG. 9D shows a dynamic ¹³C MRI of hyperpolarized [1-¹³C] NAC-[1-¹³C] NAC at pH=7 on 3T NMR spectrometer (D);

FIG. 10A shows a dynamic ¹³C MRI of hyperpolarized [1-¹³C] NAC-GSH at pH=7 on 1T NMR spectrometer (A);

FIG. 10B shows a thermal spectra of [1-¹³C] NAC-GSH was observed on 1T NMR with 43000 scans, 5 mM ProHance (B);

FIG. 10C shows a decay of dynamic ¹³C MR signal of hyperpolarized [1-¹³C] NAC-GSH on 1T NMR spectrometer (C);

FIG. 10D shows a dynamic ¹³C MRI of hyperpolarized [1-¹³C] NAC-GSH at pH=7 on 3T NMR spectrometer (D);

FIG. 11 shows a NAC (A) and its reaction products, NAC-GSH (B), have pH dependence of ¹³C chemical shifts on well optimized shimming conditions;

FIG. 12 shows an evaluation of synthesized [1-¹³C] NAC. (A)¹H NMR (left) and ¹³C NMR (right), (B) Separation and analysis on RP-HPLC (top) and ESI-MS (bottom).

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or.” The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in any suitable order unless otherwise indicated herein or otherwise clearly contradicted by context. The use of any and all examples, or exemplary language (e.g., “such as”), is intended for illustration and does not pose a limitation on the scope of the disclosure unless otherwise claimed. No language in the specification should be construed as indicating any non-claimed element as essential to the practice of the invention. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

The opened ended term “comprising” includes the intermediate and closed terms “consisting essentially of” and “consisting of.”

The term “substituted” means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example, a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

Suitable groups that may be present on an “optionally substituted” position include, but are not limited to, e.g., halogen, cyano, hydroxyl, amino, nitro, oxo, azido, alkanoyl (such as a C₂-C₆ alkanoyl group such as acyl or the like (—(C═O)alkyl)); carboxamido; alkylcarboxamide; alkyl groups, alkoxy groups, alkylthio groups including those having one or more thioether linkages, alkylsulfinyl groups including those having one or more sulfinyl linkages, alkylsulfonyl groups including those having one or more sulfonyl linkages, mono- and di-aminoalkyl groups including groups having one or more N atoms, all of the foregoing optional alkyl substituents may have one or more methylene groups replaced by an oxygen or —NH—, and have from about 1 to about 8, from about 1 to about 6, or from 1 to about 4 carbon atoms, cycloalkyl; phenyl; phenylalkyl with benzyl being an exemplary phenylalkyl group, phenylalkoxy with benzyloxy being an exemplary phenylalkoxy group. Alkylthio and alkoxy groups are attached to the position they substitute by the sulfur or oxygen atom respectively.

A dash (“-”) and (“ [symbol] ”) that is not between two letters or symbols is used to indicate a point of attachment for a substituent.

“Pharmaceutical compositions” means compositions comprising at least one active agent, such as a compound or salt of Formula (I), and at least one other substance, such as a carrier. Pharmaceutical compositions meet the U.S. FDA's GMP (good manufacturing practice) standards for human or non-human drugs.

“Carrier” means a diluent, excipient, or vehicle with which an active compound is administered. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any cancer symptom, slow cancer progression or cause cancer regression. In certain embodiments treatment of the cancer may be commenced before the patient presents symptoms of the disease.

A “therapeutically effective amount” of a pharmaceutical composition means an amount effective, when administered to a patient, to provide a therapeutic benefit such as an amelioration of symptoms, decrease cancer progression, or cause cancer regression.

A significant change is any detectable change that is statistically significant in a standard parametric test of statistical significance such as Student's T-test, where p<0.05.

### Chemical Description

Compounds disclosed may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, tautomers, polymorphs, free compound and salts) of an active agent may be employed either alone or in combination.

The term “chiral” refers to molecules, which have the property of non-superimposability of the mirror image partner.

“Stereoisomers” are compounds, which have identical chemical constitution, but differ with regard to the arrangement of the atoms or groups in space.

A “diastereomer” is a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column.

“Enantiomers” refer to two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process.

Deuteration refers to the substitution of a deuterium for hydrogen in the molecule of interest, and can be useful for increasing the T1 relaxation time of carbons in an MRI study.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

“Tautomers” or “tautomeric forms” are constitutional isomers that readily interconvert, commonly by the migration of a hydrogen atom combined with a switch of a single bond and a double bond.

“Pharmaceutically acceptable salts” include derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editors, Wiley-VCH, 2002.

The composition may further include at least one pharmaceutically acceptable excipient. A pharmaceutically acceptable excipient, as used herein, refers to a non-active pharmaceutical ingredient (“API”) substance such as a disintegrator, a binder, a filler, and a lubricant used in formulating pharmaceutical products. Each of these substances is generally safe for administering to humans according to established governmental standards, including those promulgated by the United States Food and Drug Administration (“FDA”).

A disintegrator, as used herein, refers to one or more of agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch, but is not limited thereto.

A binder, as used herein, refers to one or more of microcrystalline cellulose, hydroxymethyl cellulose, and hydroxypropylcellulose, but is not limited thereto.

A filler, as used herein, refers to one or more of calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol, but is not limited thereto.

A lubricant, as used herein, refers to one or more of agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate, but is not limited thereto.

The composition according to the present invention may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In an embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the embodiments, the composition according to the present invention can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The composition according to the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the composition according to the present invention in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The composition according to the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

The composition according to the present invention may be administered in an effective amount. An “effective amount” means an amount sufficient to show a meaningful benefit in a patient. Effective amounts may vary depending upon the biological effect desired in a patient, condition to be treated, and/or the specific characteristics of the composition according to the present invention and the individual. In this respect, any suitable dose of the composition can be administered to the patient (e.g., human), according to the biological effect desired or the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (for e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (for e.g., about 0.75 mg/kg, about 5 mg/kg, about 50 mg/kg, about 100 mg/kg, or about 200 mg/kg), about 10 mg/kg to about 200 mg/kg (for e.g., about 25 mg/kg, about 75 mg/kg, or about 150 mg/kg), or about 50 mg/kg to about 100 mg/kg (for e.g., about 60 mg/kg, about 70 mg/kg, or about 90 mg/kg).

As indicated above, the present invention is directed to a method of diagnosing or monitoring a patient suffering from cancer, the method comprising: (1) administering a pharmaceutical composition comprising an effective amount of an active agent, wherein the active agent is [1-¹³C] N-acetyl cysteine, a deuterated derivative thereof, a pharmaceutically acceptable salt of any of the foregoing thereof, or a combination thereof, together with a pharmaceutically acceptable carrier to the patient; and (2) diagnosing or monitoring the patient by hyperpolarized ¹³C-MRI. Each of these steps is discussed in more detail below.

In an embodiment, N-acetyl cysteine (NAC), the acetylated derivative of the amino acid L-cysteine and a precursor of glutathione as a promising is used as a novel probe to monitor redox status.

In an embodiment, the probe to monitor redox status includes a stable ¹³C isotope labeled NAC with a long lifetime (T₁ spin lattice relaxation) of hyperpolarization.

In an embodiment, the dose of the composition according to the present invention desirably comprises about 0.1 millimole (mmol) per kilogram (kg) of the body weight of the patient (mmol/kg) to about 10 mmol/kg (for e.g., about 0.1 mmol/kg, about 0.5 mmol/kg, about 1 mmol/kg, about 1.5 mmol/kg, about 2 mmol/kg/kg, about 2.5 mmol/kg/kg, about 3 mmol/kg, about 4 mmol/kg, about 5 mmol/kg, about 6 mmol/kg, about 7 mmol/kg, about 8 mmol/kg, about 9 mmol/kg, or about 10 mmol/kg).

In an embodiment, the dose of the composition according to the present invention desirably comprises about 0.1 mg per kilogram (kg) of the body weight of the patient (mg/kg) to about 400 mg/kg (for e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (for e.g., about 0.75 mg/kg, about 5 mg/kg, about 50 mg/kg, about 100 mg/kg, or about 200 mg/kg), about 10 mg/kg to about 200 mg/kg (for e.g., about 25 mg/kg, about 75 mg/kg, or about 150 mg/kg), or about 50 mg/kg to about 100 mg/kg (for e.g., about 60 mg/kg, about 70 mg/kg, or about 90 mg/kg).

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

The active agent may be administered singularly (i.e., sole diagnosing or monitoring agent) to diagnose or monitor a patient suffering from cancer or may be administered in combination with another agents. One or more additional diagnosing or monitoring agents along with the active agent may be administered in coordination with a pharmaceutically acceptable carrier and a regime of one or more other chemotherapeutic agents such as an antineoplastic drug, e.g., an alkylating agent (e.g., mechloroethamine, chlorambucil, cyclophosamide, melphalan, or ifosfamide), an antimetabolite such as a folate antagonist (e.g., methotrexate), a purine antagonist (e.g. 6-mercaptopurine) or a pyrimidine antagonist (e.g., 5-fluorouracil). Other, non-limiting examples of chemotherapeutic agents that might be used in coordination with one or more diagnosing or monitoring agents along with the active agent include taxanes and topoisomerase inhibitors. In addition, other non-limiting examples of active therapeutics include biological agents, such as monoclonal antibodies or IgG chimeric molecules, that achieve their therapeutic effect by specifically binding to a receptor or ligand in a signal transduction pathway associated with cancer (e.g. therapeutic antibodies directed against CD20 (e.g. rituximab) or against VEGF (e.g. bevacizumab)).

Methods of diagnosing or monitoring provided herein are also useful for treatment of mammals other than humans, including for veterinary applications such as to treat horses and livestock e.g. cattle, sheep, cows, goats, swine and the like, and pets (companion animals) such as dogs and cats.

For diagnostic or research applications, a wide variety of mammals will be suitable subjects including rodents (e.g. mice, rats, hamsters), rabbits, primates and swine such as inbred pigs and the like. Additionally, for in vitro applications, such as in vitro diagnostic and research applications, body fluids (e.g., blood, plasma, serum, cellular interstitial fluid, saliva, feces and urine) and cell and tissue samples of the above subjects will be suitable for use.

Methods of diagnosing or monitoring include providing certain dosage amounts of an active agent to a patient. Dosage levels of each active agent of from about 0.1 millimole (mmol) per kilogram (kg) of the body weight of the patient (mmol/kg) to about 10 mmol/kg per day are useful in the methods of diagnosing or monitoring (for e.g., about 0.1 mmol/kg, about 0.5 mmol/kg, about 1 mmol/kg, about 1.5 mmol/kg, about 2 mmol/kg/kg, about 2.5 mmol/kg/kg, about 3 mmol/kg, about 4 mmol/kg, about 5 mmol/kg, about 6 mmol/kg, about 7 mmol/kg, about 8 mmol/kg, about 9 mmol/kg, or about 10 mmol/kg). Dosage levels of each active agent of from about 0.1 mg to about 400 mg per kilogram of body weight per day are useful in the methods of diagnosing or monitoring (for example, about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of the active agents are provided daily to a patient. Frequency of dosage may also vary depending on the compound used and the particular diagnosing or monitoring methods used. However, for most diagnosing or monitoring methods, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

In an embodiment, the invention provides a method of diagnosing or monitoring a patient suffering from cancer, the method including administering a pharmaceutical composition comprising an effective amount of an active agent, wherein the active agent is [1-¹³C] N-acetyl cysteine, a deuterated derivative thereof, a pharmaceutically acceptable salt of any of the foregoing thereof, or a combination thereof, together with a pharmaceutically acceptable carrier to the patient; and diagnosing or monitoring the patient by hyperpolarized ¹³C-MRI. The active agent provided herein may be administered alone, or in combination with one or more other active agent.

In an embodiment, the active agent is a hyperpolarized active agent.

In an embodiment, [1-¹³C] N-acetyl cysteine is a hyperpolarized [1-¹³C] N-acetyl cysteine.

In an embodiment, the deuterated derivative is a hyperpolarized L-cysteine-[1-¹³C-2,3,3-d₃].

In an embodiment, the diagnosing or monitoring step may further include procuring a cell dynamic ¹³C-nuclear magnetic resonance (NMR) spectra of the hyperpolarized active agent at NMR spectrometer, procuring a pure phantom sample ¹³C-NMR spectrum, and comparing the procured spectrum of the hyperpolarized active agent with the pure phantom sample ¹³C-NMR spectrum.

In an embodiment, the diagnosing step may include preparing a polarizing solution of about 2 molar to about 5 molar of the active agent by titrating the polarizing solution to a pH of about 6.5 to about 7.8 using a base. For example, a polarizing solution of the active agent of about 2.5 molar to about 4.5 molar, about 3 molar to about 4 molar.

In an embodiment, the diagnosing step may include preparing a polarizing solution of about 3.2 molar active agent with a neutral pH.

In an embodiment, the diagnosing step may include a polarizing solution that remains stable overtime at both neutral and acidic pH.

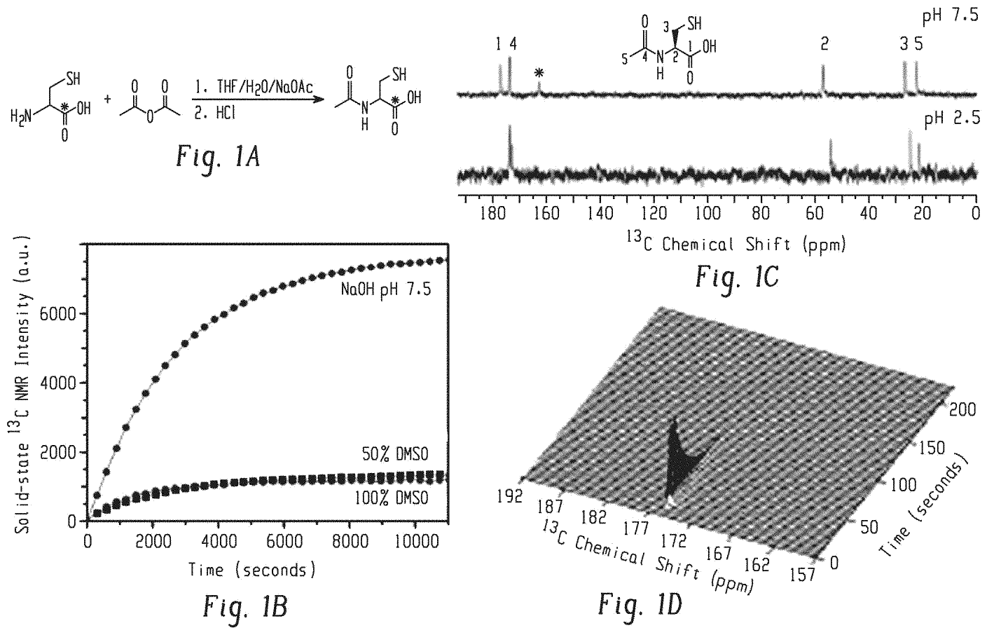

In an embodiment, the polarizing solution shows efficient polarization build-up, reaching half of the equilibrium polarization in about 10000 seconds to about 15000 seconds. For example, about 10500 seconds to about 14500 seconds, about 11000 seconds to about 14000 seconds, about 11500 seconds to about 13500, about 12000 seconds to about 13000 seconds. In an embodiment, as shown in FIG. 1B, the polarizing solution shows efficient polarization build-up, reaching half of the equilibrium polarization in 11000 seconds, making it a suitable candidate for clinical use.

As shown in FIG. 1B. Hyperpolarization build-up curves of [1-¹³C] NAC show a drastic improvement of polarization using the optimized condition of a NaOH solution at about pH 7.5 versus conventional DMSO solutions. Without wishing to be bound by a theory Applicants believe that samples with a standard solvent of DMSO polarized poorly (FIG. 1B), possibly because the anhydrous solvent favors the formation of intermolecular hydrogen bonds between two protonated carboxylic acid, which increase the dipolar coupling associated with the carbonyl carbon and shorten T₁ relaxation.

As shown in FIG. 1C, a ¹³C NMR spectra of unlabeled NAC at 1T NMR illustrates the pH dependence of polarization (a peak with an asterisk (*) is from a referencing standard of ¹³C Urea). The polarization was much weaker at pH 2.5, without wishing to be bound by a theory Applicants believe that hydrogen bonds among NAC clusters play a role in reducing the equilibrium polarization. In an embodiment, as shown in FIG. 1C, the polarizing solution remains stable overtime at both neutral and acidic pH.

In an embodiment, the diagnosing step may include T₁ relaxation time at 3T of the 3.2 molar active agent solution of about 10 seconds to 25 seconds by a decay dynamics of ¹³C magnetic resonance signal. For example, about 12 to about 22 seconds, about 15 to about 20 seconds. As shown in FIG. 1D, in an embodiment, the dynamic spectra of 3.2 molar [1-¹³C] NAC solution at 3T MRI may indicate a T₁ relaxation time of about 19.6 seconds.

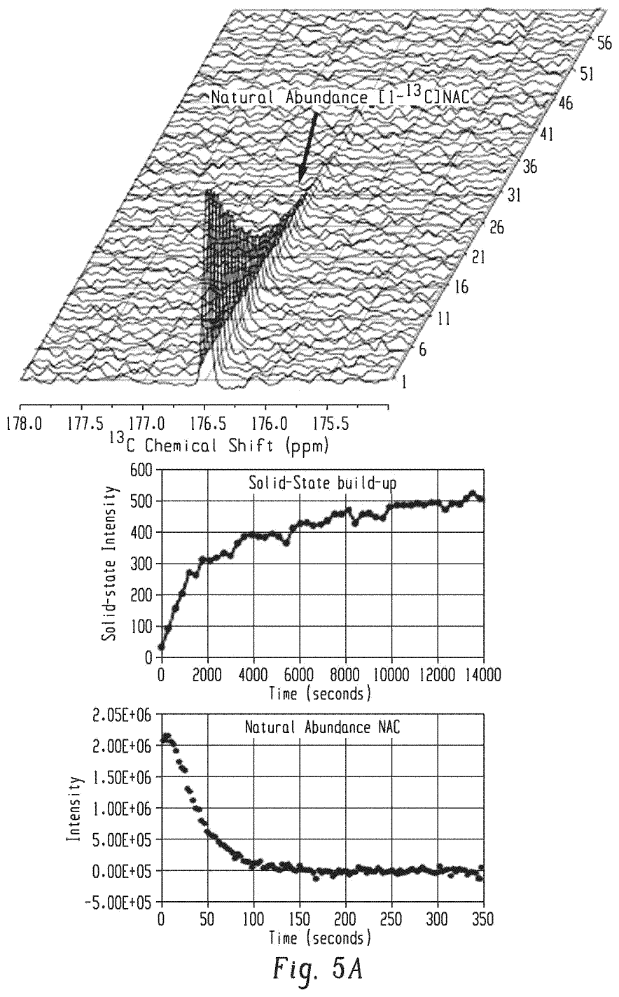

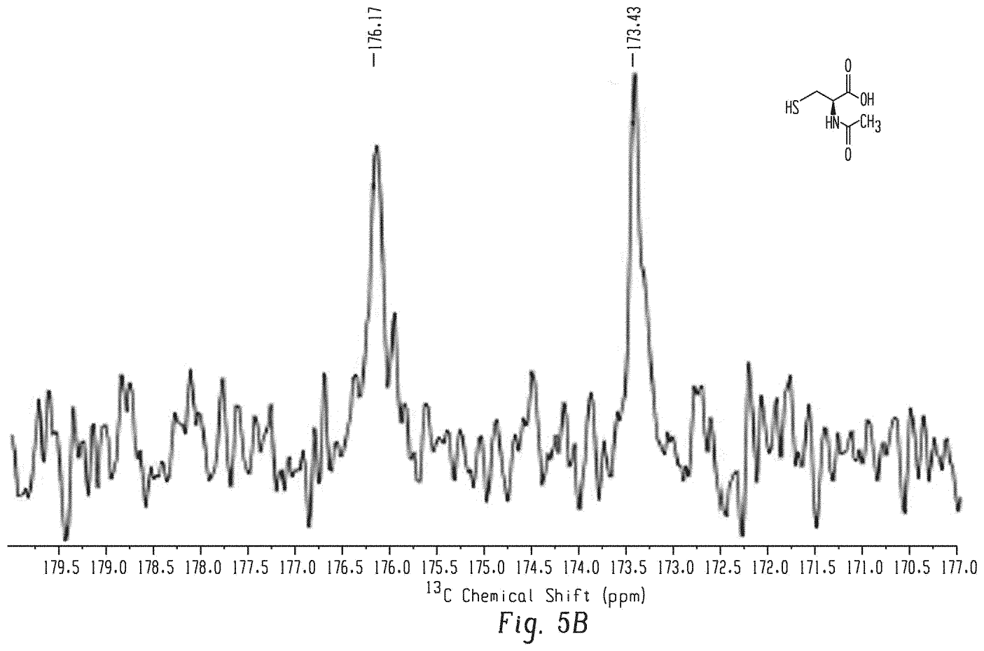

Even though NAC has two carbonyl groups in the chemical structure, including [1-¹³C] and [4-¹³C], as shown in the thermal NMR of natural abundance NAC at 1T NMR spectrometer of FIG. 5B. The hyperpolarized NMR experiments on natural abundance NAC indicated that only the [1-¹³C] NAC peak can be observed out of two potentially detectable carbonyl groups in NAC structure as shown in FIG. 5A. Without wishing to be bound by a theory inventors believe that the scalar relaxation from adjacent ¹⁴N-nuclei shortens both the T₁ and T₂ relaxation times of the [4-¹³C] peak.

In an embodiment, the active agent may be used in cell NMR and in vivo MRI.

In an embodiment, a sensitivity enhancement increase via hyperpolarization may be about 10³ to about 10⁷ fold, for example, about 10⁴ to about 10⁶ fold, about 10⁴ to about 10⁵ fold, about 10⁵ to about 10⁷ fold. In an embodiment, a sensitivity enhancement increase via hyperpolarization may be about 10⁵ fold.

In an embodiment, the cell dynamic ¹³C-nuclear magnetic resonance (NMR) spectra of the hyperpolarized [1-¹³C] N-acetyl cysteine at NMR spectrometer on a cancer cell lines may show peaks in regions about 170 ppm to 185 ppm. In an embodiment, the cancer cell line may be a human pancreatic ductal adenocarcinoma (PDAC) cell lines.

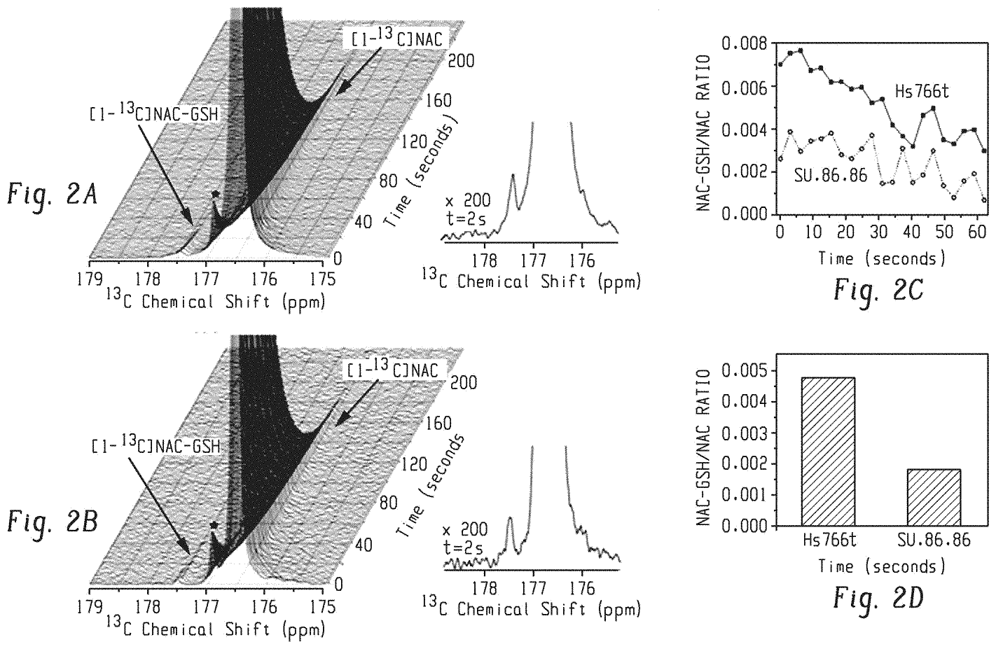

In an embodiment, a cell dynamic ¹³C NMR spectra of hyperpolarized [1-¹³C] NAC at 1T NMR spectrometer on human pancreatic ductal adenocarcinoma (PDAC) cell lines, which have one of the worst prognosises among common cancers and need effective diagnostic approaches, Hs766t (FIG. 2A) and SU.86.86 (FIG. 2B), in both cases, the cell dynamic ¹³C-nuclear magnetic resonance (NMR) spectra of the hyperpolarized [1-¹³C] N-acetyl cysteine may show three distinct peaks, a major peak at about 176.5 ppm and two peaks at about 176.8 and about 177.5 ppm.

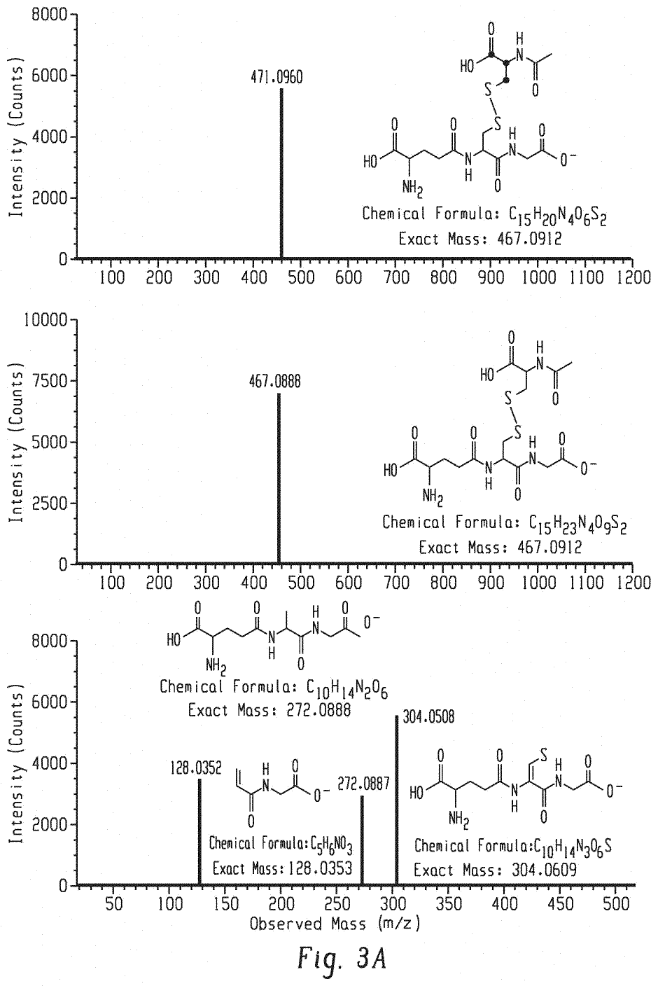

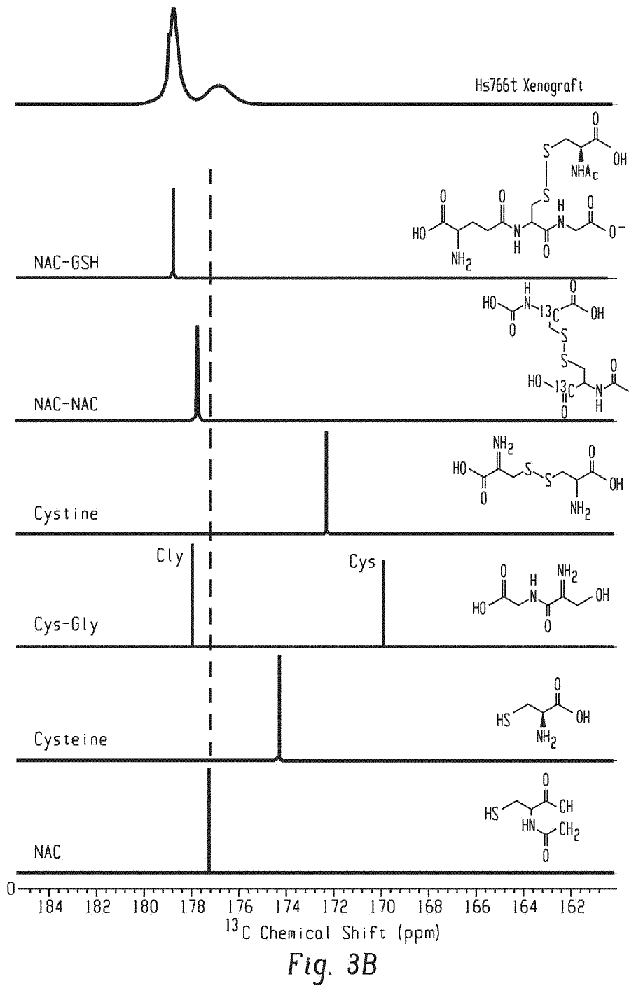

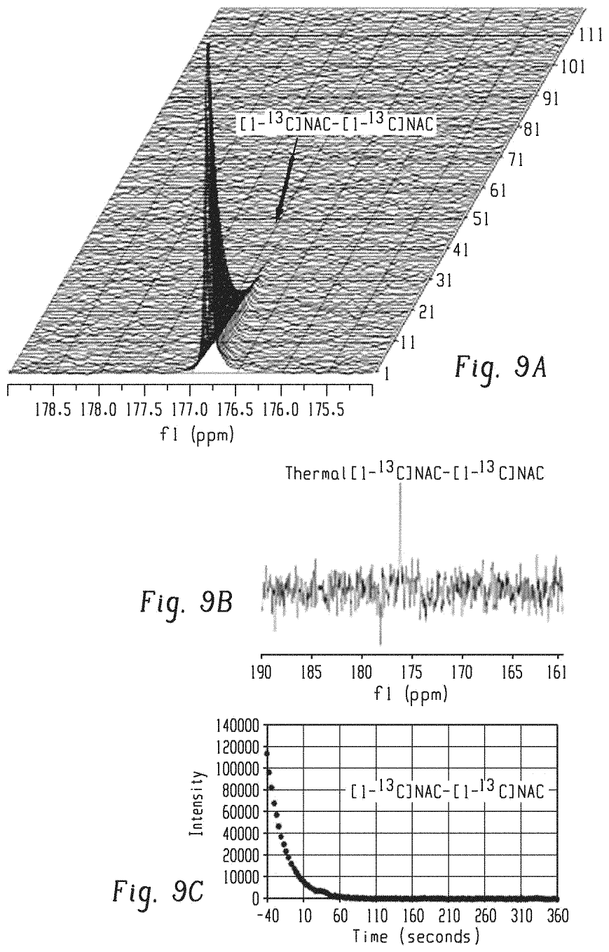

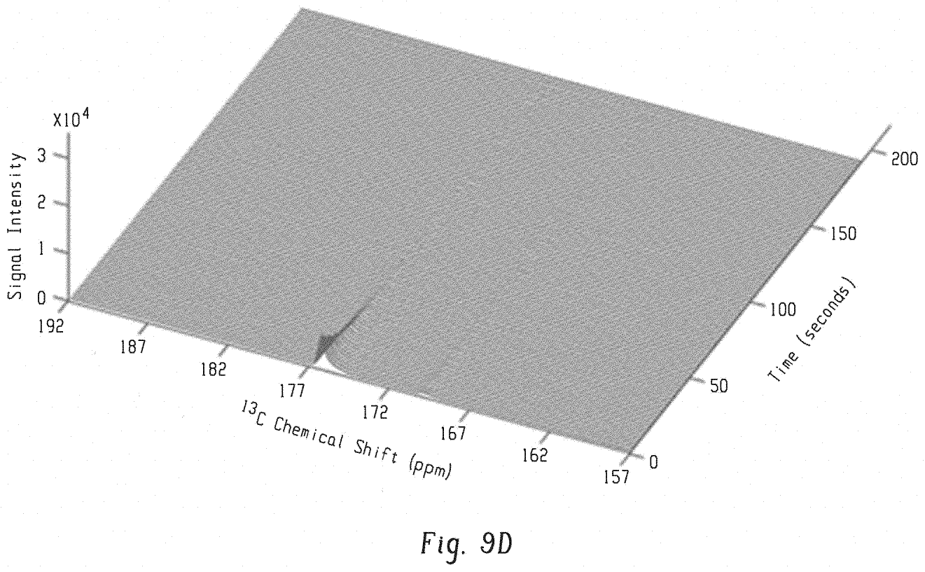

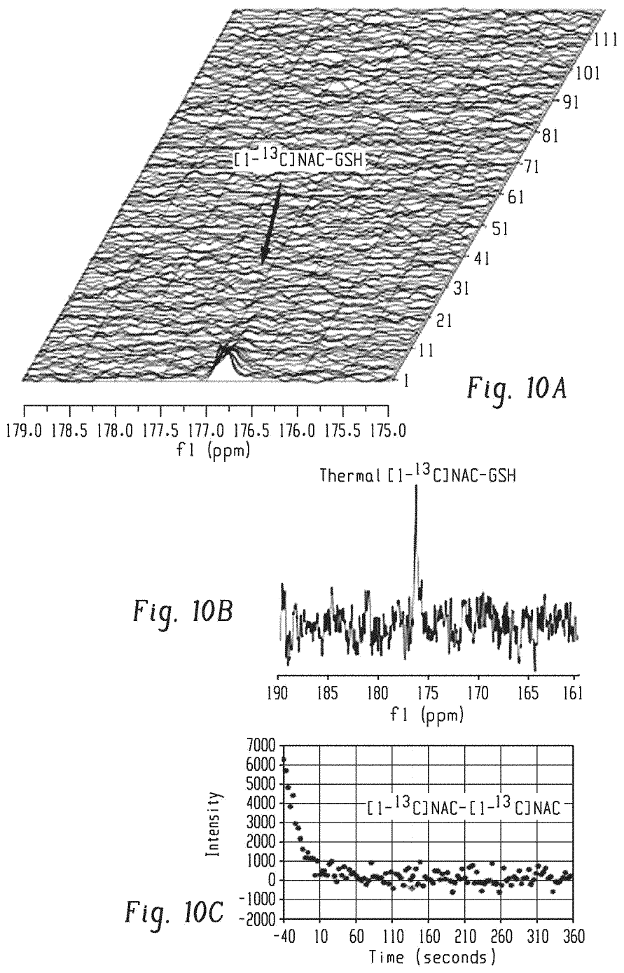

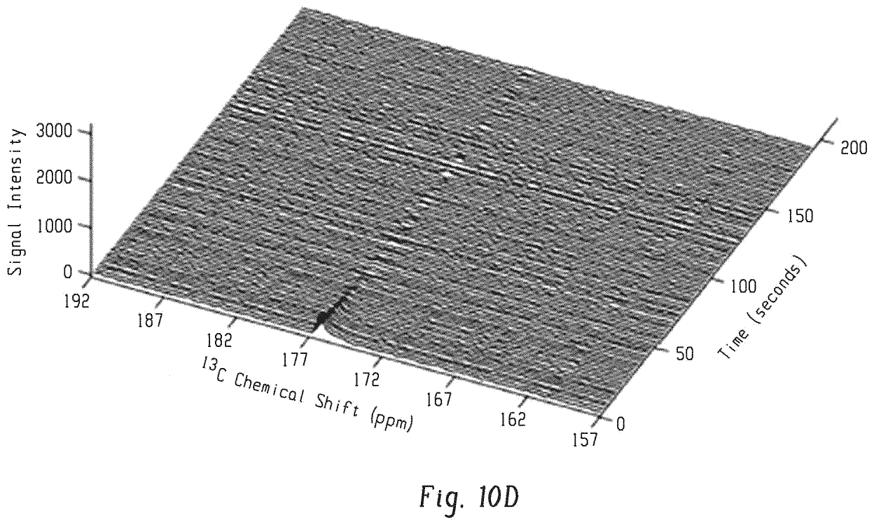

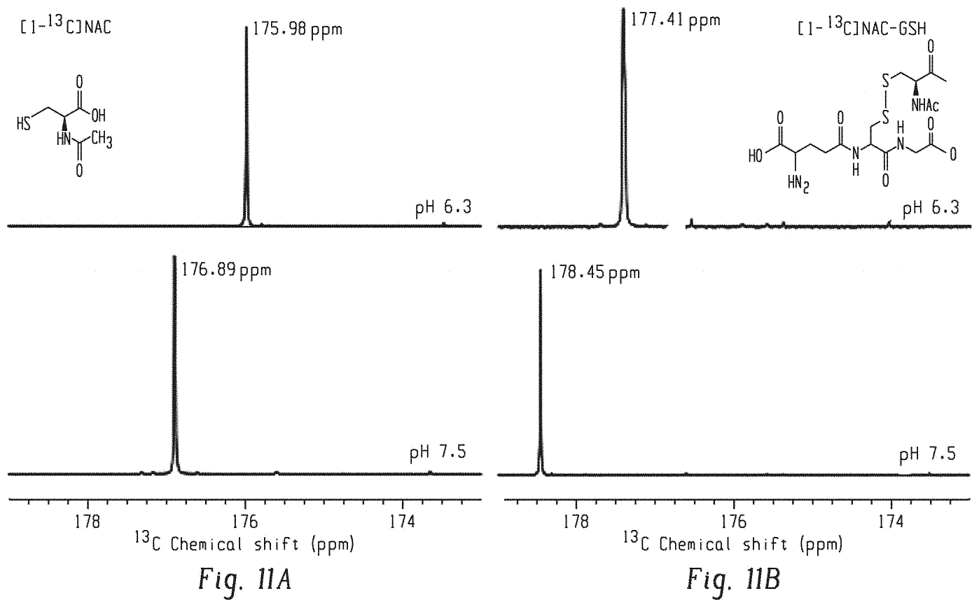

The major peak at about 176.5 ppm may be assigned to [1-¹³C] NAC on the basis of the ¹³C NMR spectrum of a pure phantom sample (FIG. 3B). The peak at about 176.8 ppm may be assigned to an oxidized NAC-NAC dimer (FIGS. 3B, 8, and 9A to 9D). The peak at about 177.4 ppm may be tentatively identified as the oxidized NAC-GSH dimer based on the ¹³C NMR spectrum of an authentic sample (FIGS. 3B, 10A to 10D).

These assignments may be confirmed by metabolomics approaches based on mass spectroscopy (MS). Tumor xenografts may be treated with unlabeled and [¹³C₃, ¹⁵N]-labeled NAC, extracted using suitable protocols and analyzed by LC/MS. The data can be collected using scanning quadrupole data-independent acquisition, which may give fragmentation information for precursor peaks to aid in identification. The NAC metabolite can be traced by first identifying retention times (rt) and m/z pairs which can be unique to the labeled sample relative to the unlabeled sample and therefore may indicate conversion products of the labeled probe as shown in FIG. 3A. As illustrated in FIG. 3B, peaks shifted by 4 Da with identical retention times correspond to labeled products. A 471/467 m/z pair with a rt of 4.47 minutes confirmed the third product at about 177.4 ppm was the oxidized NAC-GSH dimer, which was further supported by fragmentation analysis as shown in FIG. 3A.

As shown in FIG. 2C, hyperpolarized [1-¹³C] NAC can produce NAC-glutathione (NAC-GSH) in cell cultures. The rapid kinetics of this reaction suggest that hyperpolarized NAC may permeabilize through cell membranes without active transport, and chemical reactions of hyperpolarized NAC with GSH can be observed within the lifetime of this hyperpolarized ¹³C probe.

In an embodiment, a method of diagnosing or monitoring a patient suffering from cancer may involve monitoring a redox status of a redox pair. For example, monitoring the redox status of glutathione/glutathione disulfide redox pair.

In an embodiment, a patient may be a human.

In an embodiment, the monitoring step may include measuring the redox status difference between a human cancer cell lines, for example, the human pancreatic cancer cell lines of Hs766t and SU.86.86.

As shown in FIGS. 2C and 2D, the time-dependence of the NAC-GSH/NAC peak intensity ratio after mixing hyperpolarized NAC with human PDAC cell lines (FIG. 2C) and the area under the curve ratio (FIG. 2D) may suggest a higher potential for NAC oxidation with glutathione in SU.86.86 cells.

The potential for NAC to be oxidized by glutathione may depend on the GSH/GSSG balance, as NAC is not oxidized by GSH. Lower concentrations NAC-GSH in SU.86.86 is consistent with previous metabolomics experiments, as the reliance of SU.86.86 on the TCA cycle depletes NAD⁺ and therefore shifts the equilibrium of the GSH/GSSG redox buffer system towards GSH. This suggests hyperpolarized ¹³C NAC can sensitively detect the difference in GSH/GSSG status between the human pancreatic cancer cell lines of Hs766t and SU.86.86, which have significantly different metabolism in hypoxia and glycolysis but differ only moderately in their GSH/GSSG ratios (0.44 for Hs766t vs 0.95 SU.86.86).

In an embodiment including an effective in vivo hyperpolarized MRI probes with an hyperpolarized active agent, the hyperpolarized active agent exhibits the following requirements: (a) suitable biocompatibility and nontoxicity, (b) organic synthesis schemes of isotope labeled probes at high yields, (c) long spin lattice T₁ relaxation times, (d) efficient nuclear spin polarizations with the high concentrations of substrates, (e) monitoring biologically or clinically relevant mechanisms of metabolic pathways and/or physiological processes, (f) rapid distributions of the hyperpolarized probes to the targeted imaging regions, (g) adequate chemical shift differences between original injected substrates and metabolic products, (h) detectable MR signals in both injected probes and the products.

Hereinafter, an embodiment will be described in detail with reference to the following examples and procedures. However, these examples are not intended to limit the purpose and scope of the one or more example embodiments.

### EXAMPLES

Abbreviations

BOC tert-butyloxycarbonyl

DCM Dichloromethane

DI Deionized

ESI Electrospray Ionization

HILIC Hydrophilic Interaction Liquid Chromatography

LC-MS Liquid Chromatography/Mass Spectrometry

L Liter

MHz Megahertz

MSD Mass Selective Detector

MRI Magnetic Resonance Imaging

μL microliters

μm micrometer

mL milliliters

mg milligrams

mm millimeters

mM millimolar

mmol millimoles

NAC [1-¹³C] N-Acetyl Cysteine

ng Nanogram

NMR Nuclear Magnetic Resonance

RP-HPLC Reverse Phase-High Performance Liquid Chromatography

TFA Trifluoroacetic acid

THF Tetrahydrofuran

TLC Thin Layer Chromatography

General Methods

All commercially available reagents were used as received unless otherwise noted. [1-¹³C] L-cysteine and D₂O were purchased from Cambridge Isotope Laboratories, Inc (Tewksbury, Mass.). NAC, formic acid and ammonium formate was purchased from Sigma-Aldrich (St. Louis, Mo.). LC-MS acetonitrile was purchased from Fisher Scientific. Liquid chromatography mass spectrometry (LC-MS) was performed on an Agilent 1200 Series Mass Spectrometer equipped with LC/MSD TrapXCl Agilent Technologies instrument. Preparative RP-HPLC analysis was performed on an Agilent 1200 Series instrument equipped with a multi-wavelength detector. ¹H and ¹³C-NMR were recorded on a Varian 400 MHz NMR spectrometer.

Liquid chromatography/mass spectrometry analysis was performed on a Waters Acquity UPLC® coupled to a Waters Xevo Q-ToF quadruple time of flight mass spectrometer operating in electrospray ionization (ESI) in negative mode. The capillary and sampling cone voltages were set to 1.5 kV and 10 V, respectively. Source and desolvation temperatures were set to 120° C. and 450° C., respectively, and the cone and desolvation gas flows were set to 50.0 and 800.0 L/hour, respectively. To maintain mass accuracy, leucine enkephalin was used at a concentration of 2 ng/mL in 50:50 acetonitrile/water containing 0.1% formic acid and injected at a rate of 10 μL/min. Data was acquired using SONAR (scanning quadrupole data-independent acquisition) in continuum mode. In low-energy MS1 mode, the quadrupole was scanned between 50-1200 m/z, with a quadrupole transmission width of ˜50 Da, with a collision cell energy of 10 eV. In high-energy MS2 mode, the collision cell energy was ramped between 20-30 Da. The analytes were separated by HILIC chromatography on an Xbridge BEH Amide (2.5 μm, 2.1×100 mm) column. Chromatographic separation was achieved with 95:5 water:acetontrile containing 10 mM ammonium formate, pH 3 (A) and 95:5 acetonitrile:water containing 10 mM ammonium formate, pH 3 (B). Gradient elution, with a flow rate of 0.340 mL/min, began at 95% B, then decreased to 50% B from 0.0 to 3.4 minutes, 50-5% B from 3.4 to 5.39 minutes, held at 5% B from 5.39 to 6.37 minutes, then returned to initial conditions (95% B) in 0.20 minutes. The column was equilibrated at 95% B for 4.43 minutes before the next injection. The column temperature was maintained at 40° C. in a column oven.

Cell Culture and Animal Studies

All of the animal experiments were conducted in compliance with the Guide for the Care and Use of Laboratory Animal Resources, and experimental protocols were approved by the Animal Care and Use Committee, National Cancer Institute (NCI-CCR-ACUC) (National Research Council, Committee for the Update of the Guide for the Care and Use of Laboratory Animals. Guide for the care and use of laboratory animals. Washington, D.C.: Institute of Laboratory Animal Resources 2011). The human pancreatic ductal adenocarcinoma (PDAC) cell lines, Hs776t, and SU.86.86 cells, were purchased from Threshold Pharmaceuticals (Redwood City, Calif.). Human pancreatic tumor inoculated mice were generated by subcutaneous injection of 3×10⁵ cells into the right hind legs of mice. Detailed conditions for cell culture and xenograft tumor development were as described previously in K. Yamamoto, J. R. Brender, T. Seki, S. Kishimoto, N. Oshima, R. Choudhuri, S. S. Adler, E. M. Jagoda, K. Saito, N. Devasahayam, P. L. Choyke, J. B. Mitchell, and M. C. Krishna, Cancer Res 2020, 80, 2087-2093, which is incorporated herein in its entirety by reference. Athymic nude mice were obtained from the Frederick Cancer Research Center, Animal Production (Frederick, Md.). Both respiration (60-90 breaths per min) and temperature (35-37° C.) were maintained at a normal physiological range and monitored continuously during the animal experiment using the adjusted anesthesia.

Extraction of Metabolites from Tumors

¹³C, ¹⁵N labeled NAC ([¹³C₃, ¹⁵N] cysteine) was purchased from Cambridge Isotope Laboratories, Inc (Tewksbury, Mass.). Unlabeled NAC was purchased from Sigma-Aldrich (St. Louis, Mo.). 2.76 mg of either ¹³C, ¹⁵N labeled NAC ([¹³C₃, ¹⁵N] cysteine) or unlabeled NAC was intravenously injected to track metabolites of NAC in xenograft tumors. Mice were euthanized in 2 minutes after the tail vein injections. The tumors were rapidly removed and flush frozen in the liquid nitrogen, then they were stored at −80° C. The metabolites were extracted from the obtained tumors using a previously reported procedure in D. R. Crooks, T. W. Fan, and W. M. Linehan, Methods. Mol. Biol. 2019, 1928, 1-27, which is incorporated herein in its entirety by reference. The resulting lyophilized aqueous metabolite extracts were used for the MS for metabolomic analysis.

### Example 1

### Synthesis of [1-¹³C] N-acetyl cysteine ([1-¹³C]-NAC, Compound 1)

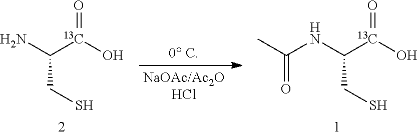

[1-¹³C] L-cysteine (Compound 2) (0.50 grams (g), 4.1 mmol) and sodium acetate trihydrate (1.11 g, 8.2 mmol) was dissolved in a degassed THF:water (90:10 v/v, 10 mL) solution and was stirred at room temperature for 20 min under nitrogen. The reaction was cooled to 0° C. and acetic anhydride (0.44 g, 4.3 mmol) was added dropwise. The reaction was stirred for 16 hours (h) at room temperature under nitrogen. The clear solution was cooled and acidified to pH 1 with concentrated HCl. The solvent was evaporated in vacuo and the product purified by RP-HPLC. Purification was performed using an Agilent Prep C18 column (5 μm, 50×100 mm) with a flow rate of 50 mL/min. A linear gradient of 5-35% acetonitrile with 0.1% TFA was used to elute the product Compound 1 as a white, hygroscopic powder after lyophilization (0.41 g, 64%). FIG. 1A also shows the synthetic scheme of [1-¹³C] NAC.

¹H-NMR (400 MHz, D₂O): δ 2.08 (3H, s, CH₃), 2.99 (2H, m, CH₂SH), 4.63 (1H, m, NHCH). ¹³C-NMR (400 MHz, D₂O): δ 23.45 (CH₃), 27.41 (CH₂SH), 57.51 (d, ¹J_{C—C}=232 Hz, NHCH), 173.66 (CH₃C═O), 176.89 (COOH). m/z (ESI-MS+): 165.0 [M+H]⁺.

### Example 2

### Hyperpolarized ¹³C MRI

35 mL of 3.2 M [1-¹³C] NAC with 17 mM OX063 was hyperpolarized using the SPINlab (GE Healthcare) for 3-4 hours, and the scans were performed using the Philips Achieva 3T MRI. ¹³C two dimensional spectroscopic chemical shift images (CSIs) were acquired with a 28×28 mm, field of view in a 10 mm axial slice through the head, a matrix size of 14×14, spectral width of 3333 Hz, repetition time of 86 ms, and excitation pulse width a flip angle of 3° for the mouse head, and with a 32×32 mm, field of view in a 10 mm coronal slice through the body, a matrix size of 16×16, spectral width of 3333 Hz, repetition time of 85 ms, and excitation pulse with a flip angle of 10⁰ for the mouse body. CSIs were acquired 30 seconds after the beginning of the hyperpolarized [1-¹³C] NAC injections.

### Example 3

### Synthesis of [1-¹³C] N-Acetyl cysteine-[1-¹³C] N-acetyl cysteine (Compound 3) and [1-¹³C]N-acetyl cysteine-Glutathione (Compound 4)

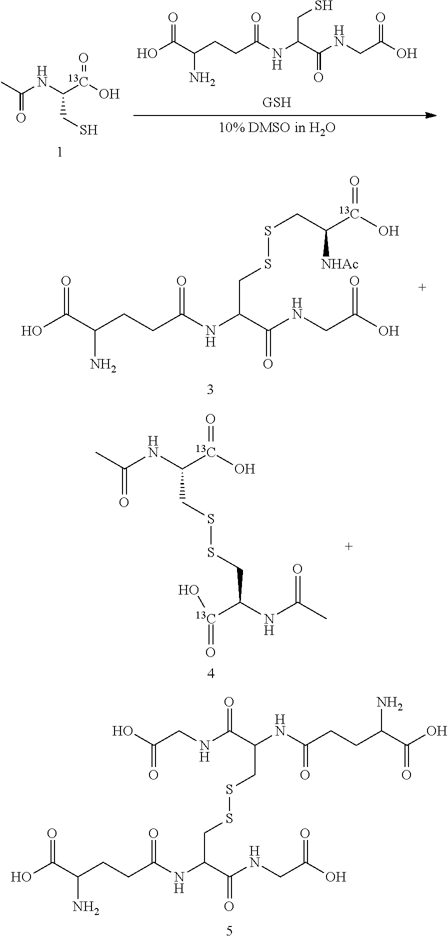

Compound 1 (0.0257 g, 0.156 mmole) and GSH (0.0962 g, 0.313 mmole) powders were combined in a vial. To this mixture, a 10% solution of DMSO in DI water (1.0 mL) was added. The resulting mixture was gently shaken for 5 minutes until a clear solution was obtained and the vial was exposed to air for 16 h at RT. LC-MS analysis indicated presence of Compound 3, Compound 4 and Compound 5 dimers. Products Compound 3 and Compound 4 were observed to be in roughly equal proportion while 5 was the major product. Reaction mixture was diluted with 20 mL DI water and lyophilized to obtain solid powder. Separation of various products was performed using an Agilent Prep C18 column (5 μm, 50×100 mm) with a flow rate of 50 mL/min. A linear gradient of 0-25% acetonitrile with 0.05% TFA was used to elute the products, all obtained as white powder after lyophilization.

Compound 3: ¹H-NMR (400 MHz, D₂O): ¹H NMR (400 MHz, D₂O) δ 4.65-4.54 (m, 1H), 3.99-3.91 (m, 2H), 3.88-3.77 (m, 1H), 3.31 (ddd, J=14.2, 6.8, 4.1 Hz, 3H), 3.10-2.91 (m, 3H), 2.55 (hept, J=7.7 Hz, 3H), 2.27-2.13 (m, 3H), 2.08 (d, J=1.1 Hz, 3H); ¹³C-NMR (100 MHz, D₂O) δ 175.72; m/z (ESI-MS+): 470.10 [M+H]⁺.

Compound 4: ¹H-NMR (400 MHz, D₂O) δ 4.70-4.63 (m, 1H), 4.59 (dd, J=6.6, 5.6 Hz, 1H), 3.98 (d, J=0.8 Hz, 2H), 3.89-3.78 (m, 1H), 3.37-3.26 (m, 1H), 3.11-2.88 (m, 3H), 2.65-2.48 (m, 2H), 2.19 (td, J=7.5, 6.3 Hz, 2H), 2.08 (d, J=0.7 Hz, 3H); ¹³C-NMR (100 MHz, D₂O) δ 175.11; m/z (ESI-MS+): 327.05 [M+H]⁺.

### Example 4

### Synthesis of [1-¹³C₂] L-Cystine (Compound 6)

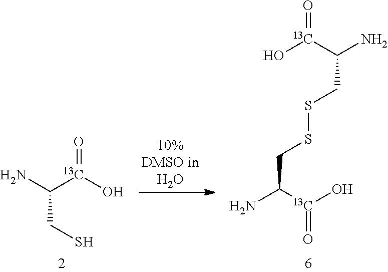 [SMILES: N[C@@H](CS)[13C](=O)O ; N[C@H](CSSC[C@H](N)[13C](=O)O)[13C](=O)O]

[1-¹³C] L-cysteine (Compound 2) (0.010 g, 0.08 mmol) was dissolved in 10% solution of DMSO in DI water (250 μl) and gently shaken for 5 minutes until a clear solution was obtained. It was then exposed to air for 16 h at RT. Analysis by LC-MS indicated a complete conversion of the starting material to [1-¹³C₂] L-Cystine (Compound 6). Reaction mixture was diluted with 5 ml of DI water and lyophilized to obtain Compound 6 a white powder.

¹H-NMR (400 MHz, D₂O) δ 4.02 (ddd, J=7.7, 6.6, 4.4 Hz, 2H), 2.96 (dt, J=15.3, 4.3 Hz, 2H), 2.82 (ddd, J=15.3, 7.8, 2.3 Hz, 2H); ¹³C-NMR (100 MHz, D₂O) δ 169.74; m/z (ESI-MS+): 243.02 [M+H]⁺.

### Example 5

### Synthesis of N-acetyl-L-cysteine-[1-¹³C-2,3,3-d₃] (Compound 12)

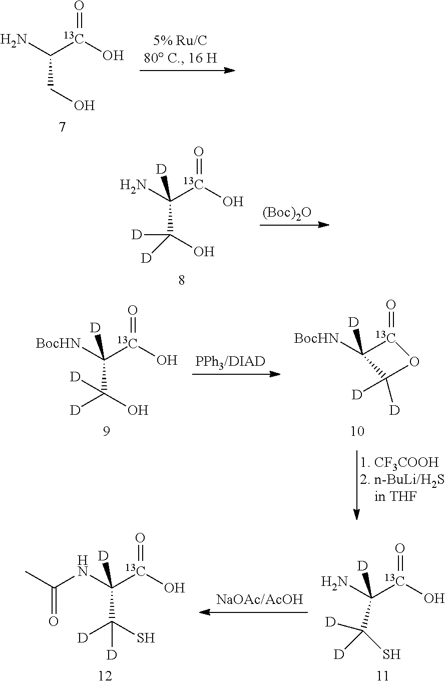

### Step 1: Synthesis of L-Serine-[1-¹³C-2,3,3-d₃] (Compound 8)

To a solution of L-Serine-[1-¹³C] (Compound 7) (1.06 g, 10 mmole) in D₂O (50 mL), ruthenium on carbon (400 mg, 5% loading) was added and the flask was sealed. Air was evacuated from the flask and replaced with D₂ gas. Reaction flask was attached with D₂ gas balloon and the reaction mixture was stirred vigorously at 80° C. for 16 h. The balloon was refilled with fresh D₂ gas and stirring was continued at 80° C. for additional 16 h. Reaction mixture was then filtered over a celite pad, residue washed with water and combined liquid phases were lyophilized. The product (Compound 8) was obtained as solid, white powder (1.06 g, 97%).

¹³C-NMR (100 MHz, D₂O) δ 172.7 (¹³C═O); HRMS (ESI/Q-TOF) m/z: [M+H]⁺ Calcd for C₂¹³CH₅D₃NO₃ 110.0726; Found 110.0723.

### Step 2: Synthesis of (tert-butoxycarbonyl)-L-serine-[1-¹³C-2,3,3-d₃](Compound 9)

To a suspension of L-serine-[1-¹³C-2,3,3-d₃] (0.59 g, 5.4 mmole) (Compound 8) and Di-tert-butyl dicarbonate (1.77 g, 8.11 mmole) in anhydrous methanol (25 mL), triethyl amine (0.754 mL, 5.4 mmole) was added dropwise. Reaction mixture was stirred at RT for 20 h. To this, 20 mL water was added and stirred at RT for 30 minutes. Solvents from the reaction mixture were removed under reduced pressure. Crude was chromatographed on NP silica using DCM-MeOH, gradient of 100% DCM→30% DCM, to yield product (Compound 9) as colorless oil (1.04 g, 92%).

¹H-NMR (400 MHz, CDCl₃) δ 7.35 (S, —NH, 1H), 1.43 (S, —CH₃, 9H); ¹³C-NMR (100 MHz, CDCl₃) δ 174.8 (¹³C═O), 156.1 (—NHCO), 80.1 (—C(CH₃)₃), 28.3 (—CH₃); HRMS (ESI/Q-TOF) m/z: [M−H]⁻ Calcd for C₇¹³CH₁₁D₃NO₅ 208.1094; Found 208.1099.

### Step 3: Synthesis of tert-butyl (S)-(2-oxooxetan-3-yl-2-¹³C-3,4,4-d₃) carbamate (Compound 10)

A suspension of triphenyl phosphine (0.262 g, 1 mmole) in anhydrous THF (4 mL) was cooled to −78° C. To this, di-isopropyl azodicarboxylate was added dropwise and stirred for 10 minutes. To this, a solution of Compound 9 (0.209 g, 1 mmole) in anhydrous THF (4 mL) was added dropwise. Reaction mixture was stirred for 20 minutes at −78° C. followed by stirring for 180 minutes at RT. Solvent from the reaction mixture was removed under reduced pressure. Crude was chromatographed on NP silica using DCM-MeOH, gradient of 100% DCM→30% DCM, to yield the product, BOC-L-Serine-β-lactone (Compound 10), as a white solid.

¹H-NMR (400 MHz, CDCl₃) δ 1.46 (S, —CH₃, 9H); ¹³C-NMR (100 MHz, CDCl₃) δ 173.8 (¹³C═O), 169.2 (—NHCO), 59.2 (—C(CH₃)₃), 28.3 (—CH₃); HRMS (ESI/Q-TOF) m/z: [M−H]-Calcd for C₇¹³CH₉D₃NO₄ 190.0988; Found 190.0991.

### Step 4: Synthesis of L-cysteine-[1-¹³C-2,3,3-d₃] (Compound 11)

In a dry flask, BOC-L-Serine-β-lactone (Compound 10) (1.0 eq.) was treated with anhydrous trifluoro acetic acid at 0° C. for 10 minutes. Excess trifluoroacetic acid and tert-butyl trifluoroacetate was removed under reduced pressure at 25° C. The resulting intermediate was used immediately in the following step. In a separate flask, a suspension of LiSH was made by adding a solution of H₂S (0.8 M in THF) into a solution of n-butyl lithium (1.6 M in Hexanes) at 0° C. To the suspension of LiSH, a solution of L-Serine-β-lactone in anhydrous acetonitrile was added dropwise and stirred for 1 h under the atmosphere of Ar. The mixture was acidified with conc. HCl (1.5 eq.) and solvents were removed in vacuo at 35° C. Crude product was purified on RP flash column using degassed water as an eluent to yield the product (Compound 11) as a white powder.

### Step 5: Synthesis of N-acetyl-L-cysteine-[1-¹³C-2,3,3-d₃] (Compound 12)

L-cysteine-[1-¹³C-2,3,3-d₃] (Compound 11) (0.50 g, 4.1 mmol) and sodium acetate trihydrate (1.11 g, 8.2 mmol) were dissolved in a degassed THF:water (90:10 v/v, 10 mL) solution and was stirred at room temperature for 20 min under nitrogen. The reaction was cooled to 0° C. and acetic anhydride (0.44 g, 4.3 mmol) was added dropwise. The reaction was stirred for 16 h at room temperature under nitrogen. The clear solution was cooled and acidified to pH 1 with concentrated HCl. The solvent was evaporated in vacuo and the product purified by RP-HPLC. Purification was performed using an Agilent Prep C18 column (5 μm, 50×100 mm) with a flow rate of 50 mL/min. Isocratic gradient of DI water with 0.05% TFA was used to elute the product (Compound 12) as a white, hygroscopic powder after lyophilization.

### NMR Analysis

Furthermore, to test the effectiveness of [1-¹³C] NAC as an imaging probe in vivo, real-time dynamic ¹³C MR spectra of hyperpolarized [1-¹³C] NAC were acquired from mice bearing tumor xenograft.

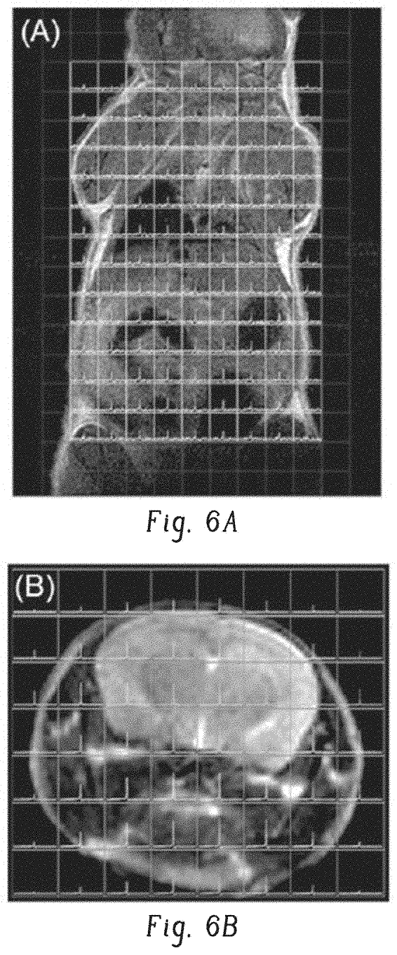

¹³C two-dimensional chemical shift imaging (CSI) experiments in both a healthy mouse body and head after intravenous (iv) injection of hyperpolarized [1-¹³C] NAC solution through a tail vein cannula as shown in FIGS. 6A and 6B. Hyperpolarized [1-¹³C] NAC was globally distributed throughout the mouse body within 30 seconds after the injection of hyperpolarized solutions, with higher concentrations of [1-¹³C] NAC in the liver, kidney, and heart region. As shown in FIG. 6A, a lower signal was observed in the lung region. Although the blood-brain barrier (BBB) permeability of NAC is subject to controversy, the presence of hyperpolarized [1-¹³C] NAC in the normal mouse brain indicates that the membrane-permeable NAC may penetrate the blood-brain barrier and be retained in the brain as shown in FIG. 6B.

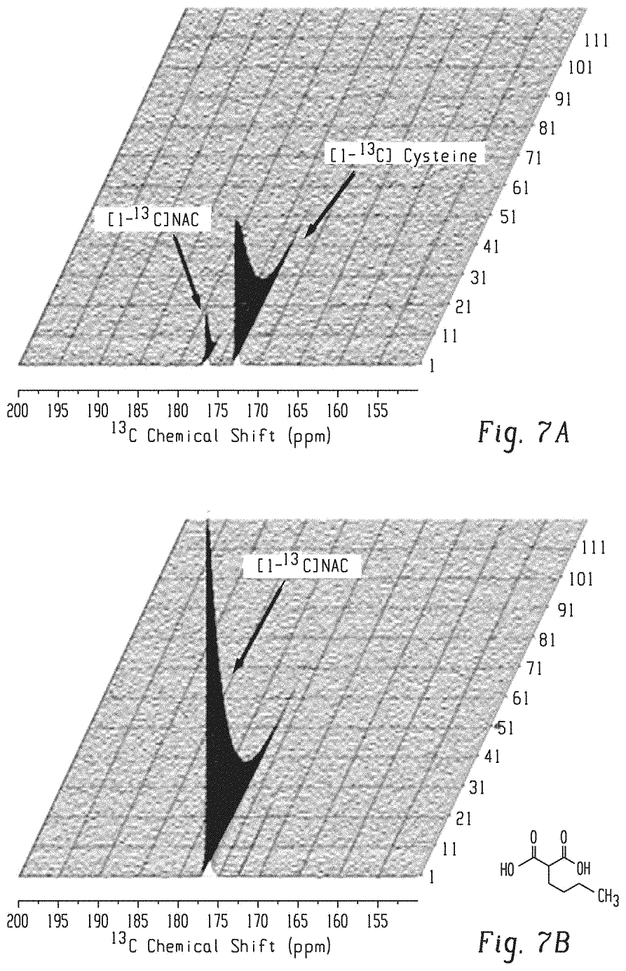

As shown in FIGS. 7A and 7B, metabolites of in vivo hyperpolarized [1-¹³C] NAC were not observed in the liver and kidney regions of these normal mice, suggesting that the enzymatic conversion of NAC was below the detection level in the absence of any imposed oxidative stress either focally or globally, although in vitro enzymatic assays of hyperpolarized NAC incubated with acylase 1 resulted in immediate production of cysteine.

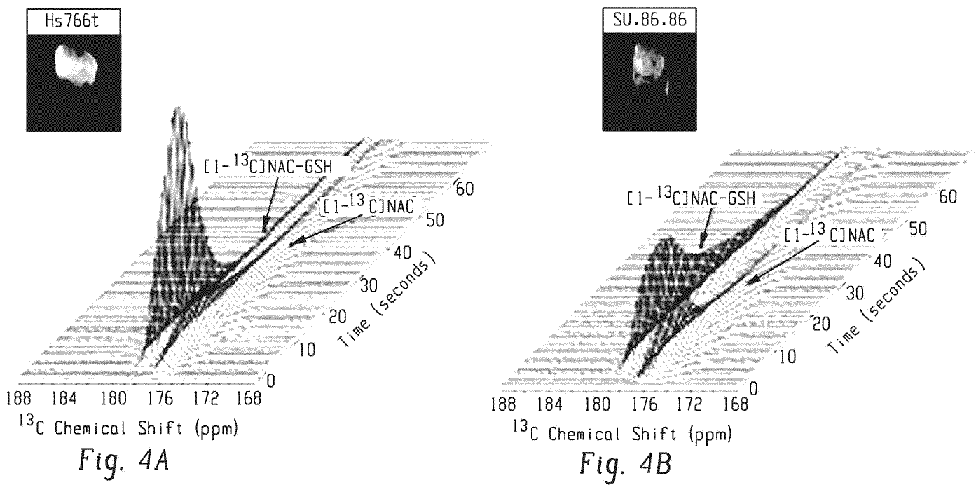

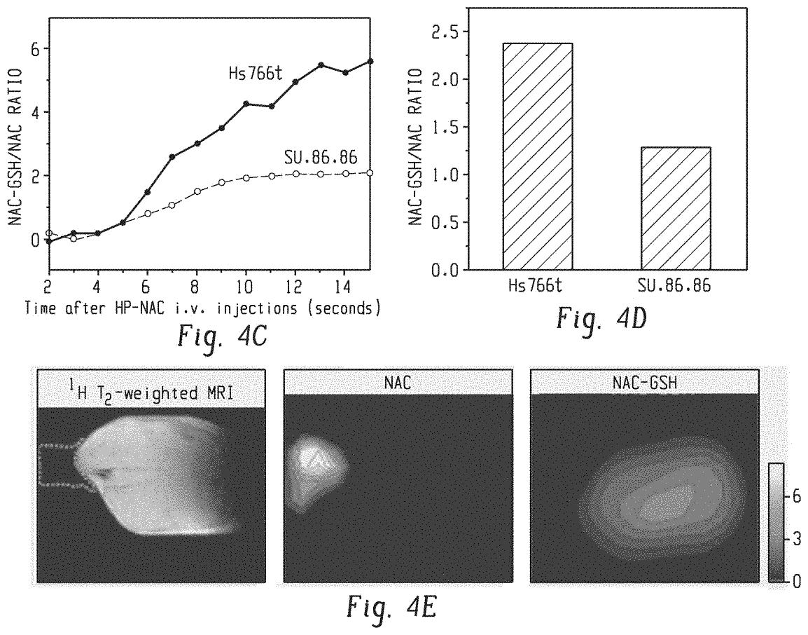

To test [1-¹³C] NAC in a tumor environment, mouse leg xenografts of Hs766t and SU.86.86 are prepared. The single voxel MRI signal for NAC-GSSG is much stronger in the xenografts (FIGS. 4A and 4B), consistent with higher cellular density in vivo. In other aspects, the in vivo data (FIGS. 4C and 4D) resembles the in vitro data of the corresponding cell cultures (FIGS. 2C and 2D). Similar to the in vitro results, NAC-GSH is rapidly formed in both tumors and the amount of NAC-GSH formed is higher in Hs766t than in SU.86.86 tumors. These results show that NAC-GSH formation could be imaged as shown in FIG. 4E. Using chemical shift imaging, it can be seen that NAC-GSH formation is highest in the tumor and lowest in the surrounding muscle and leg regions while the distribution of non-converted NAC was observed dominantly in the leg area, which is consistent with higher overall glutathione concentrations in the tumor regions (FIG. 4E).

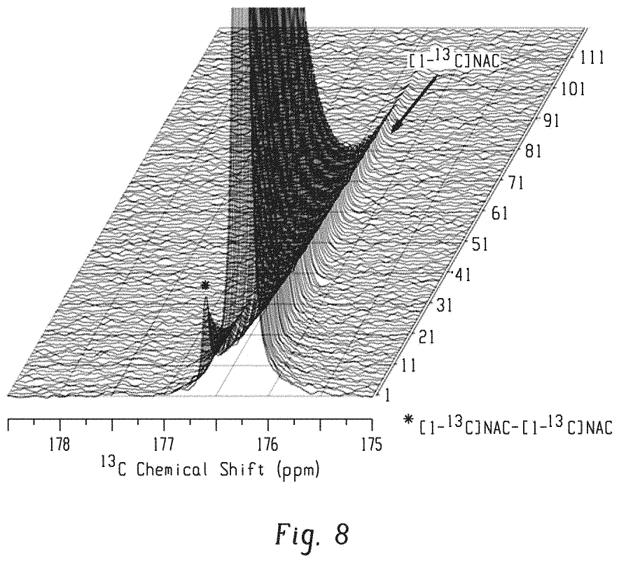

FIG. 8 shows a dynamic ¹³C MRI of hyperpolarized [1-¹³C] NAC on 1T NMR spectrometer, in addition to major [1-¹³C] NAC peak, the minor signal of [1-¹³C] NAC-[1-¹³C] NAC was observed with optimized shimming conditions.

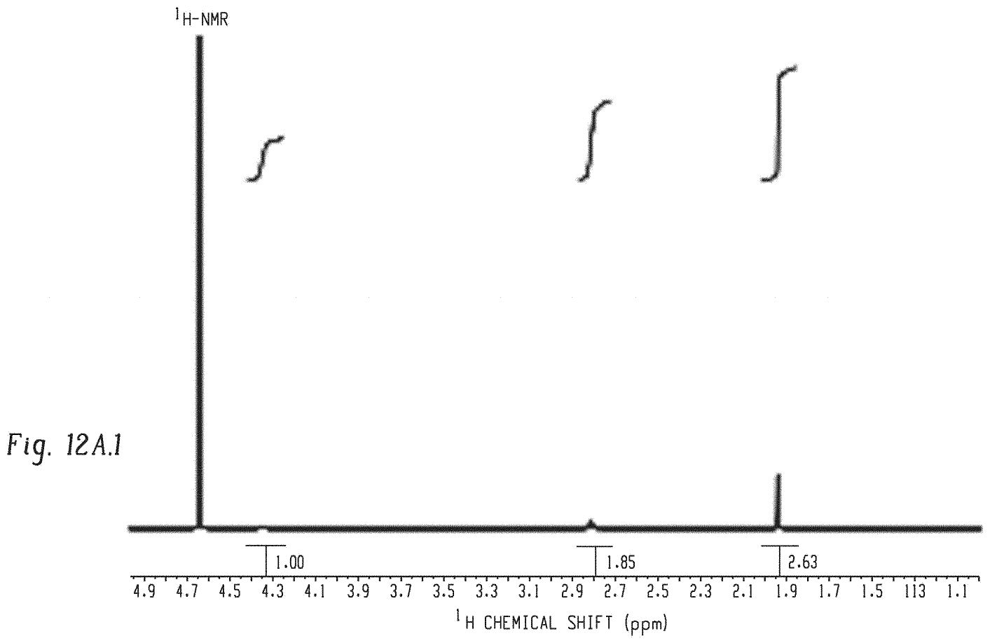

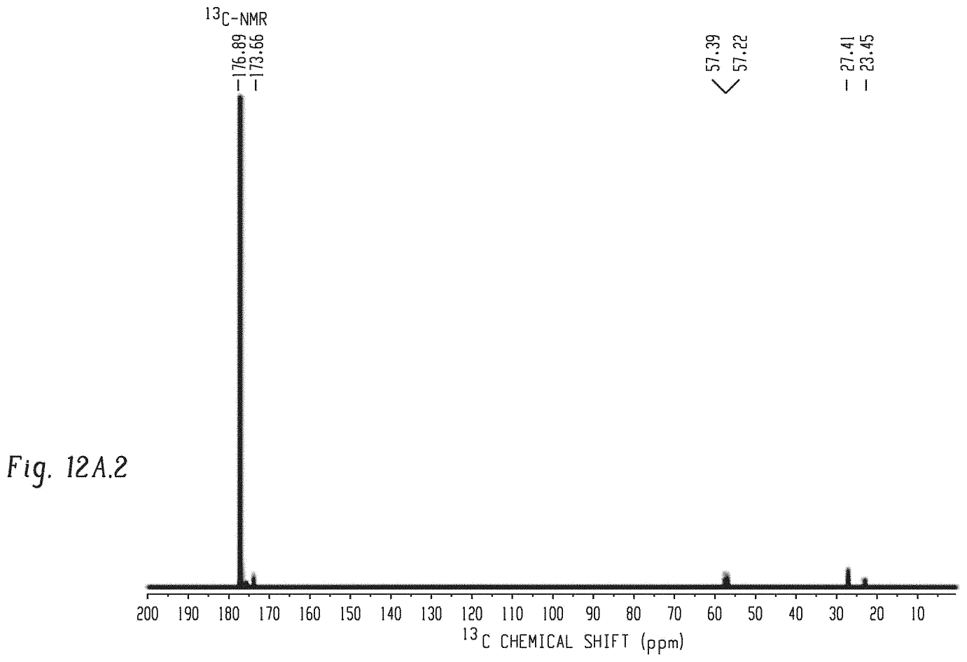

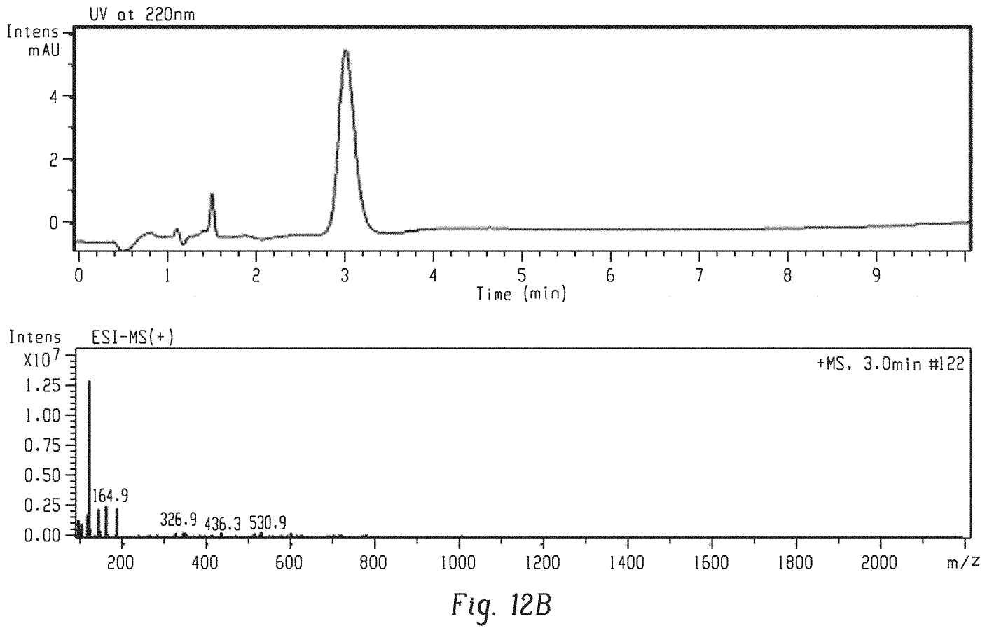

As shown in FIGS. 11 and 12, both NAC and its reaction products, NAC-GSH, have pH dependence of ¹³C chemical shifts at high field magnet and/or well optimized shimming conditions.

While this disclosure has been described in connection with what is presently considered to be practical example embodiments, it is to be understood that the disclosure is not limited to the disclosed exemplary embodiments, but, on the contrary, is intended to cover various modifications and equivalent arrangements included within the spirit and scope of the appended claims.

1. A method of diagnosing or monitoring a patient suffering from cancer, the method comprising:
administering a pharmaceutical composition comprising an effective amount of an active agent, wherein the active agent is [1-¹³C] N-acetyl cysteine, a deuterated derivative thereof, a pharmaceutically acceptable salt of any of the foregoing thereof, or a combination thereof, together with a pharmaceutically acceptable carrier to the patient; and
diagnosing or monitoring the patient by hyperpolarized ¹³C-MRI.

2. The method of claim 1, wherein the active agent is a hyperpolarized active agent.

3. The method of claim 1, wherein said [1-¹³C] N-acetyl cysteine is a hyperpolarized [1-¹³C] N-acetyl cysteine.

4. The method of claim 1, wherein said deuterated derivative is hyperpolarized L-N-acetyl cysteine-[1-¹³C-2,3,3-d₃].

5. The method of claim 1, wherein said diagnosing or monitoring step further comprises:
procuring a cell dynamic ¹³C-nuclear magnetic resonance (NMR) spectra of the hyperpolarized active agent at NMR spectrometer,
procuring a pure phantom sample ¹³C-NMR spectrum, and
comparing the procured spectrum of the hyperpolarized active agent with the pure phantom sample ¹³C-NMR spectrum.

6. The method of claim 1, wherein said diagnosing step comprises preparing a polarizing solution of about 2 molar to about 5 molar the active agent by titrating the polarizing solution to a pH of about 6.5 to about 7.8 using a base.

7. The method of claim 6, wherein the polarizing solution used is about 3.2 molar active agent solution.

8. The method of claim 6, wherein said polarizing solution remains stable overtime at both neutral and acidic pH.

9. The method of claim 8, wherein the polarizing solution build-up time reaching half of the equilibrium polarization in about 10000 seconds to about 15000 seconds.

10. The method of claim 9, wherein the polarizing solution build-up time reaching half of the equilibrium polarization in about 11000 seconds.

11. The method of claim 1, wherein said diagnosing step comprises T₁ relaxation time at 3T of the 3.2 molar active agent solution of about 10 seconds to 25 seconds by a decay dynamics of ¹³C magnetic resonance signal.

12. The method of claim 11, wherein said diagnosing step comprises T₁ relaxation time at 3T of the 3.2 molar active agent solution of about 15 seconds to 20 seconds by a decay dynamics of ¹³C magnetic resonance signal.

13. The method of claim 12, wherein said diagnosing step comprises T₁ relaxation time at 3T of a 3.2 molar [1-¹³C] NAC solution of about 19.6 seconds by a decay dynamics of ¹³C magnetic resonance signal.

14. The method of claim 1, wherein said diagnosing step comprises a sensitivity enhancement increase via hyperpolarization of about 10³ to about 10⁷ fold.

15. The method of claim 14, wherein said diagnosing step comprises a sensitivity enhancement increase via hyperpolarization of about 10⁵ fold.

16. The method of claim 1, wherein said diagnosing step comprises a cell dynamic ¹³C-nuclear magnetic resonance (NMR) spectra of the hyperpolarized [1-¹³C] N-acetyl cysteine at NMR spectrometer on a cancer comprising peaks in regions about 170 ppm to 185 ppm.

17. The method of claim 16, wherein the cancer is human pancreatic ductal adenocarcinoma (PDAC).

18. The method of claim 17, wherein the cell dynamic ¹³C-nuclear magnetic resonance (NMR) spectra of the hyperpolarized [1-¹³C] N-acetyl cysteine on human pancreatic ductal adenocarcinoma (PDAC) comprises three peaks, a major peak at about 176.5 ppm and two peaks at about 176.8 and at about 177.5 ppm.

19. The method of claim 1, wherein said diagnosing step comprises permeabilizing the hyperpolarized active agent through cell membranes without an active transport.

20. The method of claim 1, wherein said monitoring step comprises monitoring a redox status of a redox pair.

21. The method of claim 1, wherein said monitoring step comprises monitoring the redox status of glutathione/glutathione disulfide redox pair.

22. The method of claim 1, wherein said monitoring step comprises measuring the redox status difference between a human cancers.

23. The method of 22, wherein said human cancer is a human pancreatic cancer.

24. (canceled)

25. The method of claim 1, wherein the patient is human.

26. The method of claim 1, wherein the composition further comprises a pharmaceutically acceptable carrier and one or more additional chemotherapeutic agents selected from an antineoplastic drug, an antimetabolite, a purine antagonist, a pyrimidine antagonist, taxanes and topoisomerase inhibitors, or biological agents.

27. A method of synthesizing [1-¹³C] N-acetyl cysteine or a deuterated derivative thereof, the method comprising:
reacting [1-¹³C]-cysteine or a deuterated derivative thereof with an acetylating agent to form [1-¹³C] N-acetyl cysteine or the deuterated derivative thereof, and
isolating [1-¹³C] N-acetyl cysteine or the deuterated derivative thereof.

28. The method of claim 27, wherein the deuterated derivative of [1-¹³C]-cysteine is L-N-acetyl-cysteine-[1-¹³C-2,3,3-d₃].

29. The method of claim 27, wherein the acetylating agent is acetic anhydride, acetyl chloride, or acetic acid.

30. The method of claim 27 wherein said reacting step comprises, converting a carboxylate salt of [1-¹³C] N-acetyl cysteine or the deuterated derivative thereof to [1-¹³C] N-acetyl cysteine or the deuterated derivative thereof.

31. The method of claim 30 wherein said carboxylate salt of [1-¹³C] N-acetyl cysteine or the deuterated derivative thereof is converted to the [1-¹³C] N-acetyl cysteine or the deuterated derivative thereof by treating with either hydrogen chloride (HCl) gas or concentrated aqueous HCl.

32. The method of claim 23 wherein said isolating step comprises, purification by high performance liquid chromatography (HPLC).

33. The method of claim 23 wherein said isolating step comprises lyophilization.
